(19) United States
(12) Patent Application Publication
(10) Pub. No.: US 2022/0304955 A1
(43) Pub. Date: Sep. 29, 2022

(54) TREATMENT OF FERROPTOSIS

(71) Applicant: Nacuity Pharmaceuticals, Inc., Fort Worth, TX (US)

(72) Inventors: G. Michael Wall, Fort Worth, TX (US); Ashley Bush, Parkville (AU); Gawain McColl, Parkville (AU); Nicole Jenkins, Parkville (AU)

(21) Appl. No.: 17/701,051

(22) Filed: Mar. 22, 2022

Related U.S. Application Data
(60) Provisional application No. 63/164,843, filed on Mar. 23, 2021.
(60) Provisional application No. 63/226,572, filed on Jul. 28, 2021.

(51) Int. Cl. A61K 31/16; A61K 45/06

(52) U.S. Cl. CPC A61K 31/16; A61K 45/06

(57) ABSTRACT

The present invention relates in general to the field of compositions and methods for using N-acetylcysteine amide (NACA) or (2R,2R′)-3,3′-disulfanediyl bis(2-acetamidopropanamide) (diNACA) for treating or alleviating a disorder or condition associated with ferroptosis, which can cause or contribute to diseases, disorders and conditions including aging associated with declining cognition, cognitive impairment, declining physical function, physical decline, elevated inflammation, endothelial dysfunction, insulin resistance, central obesity, loss of muscle mass (sarcopenia), neurological diseases and disorders, including neurodegeneration, stroke, including ischemic stroke and post-hemorrhagic stroke damage, and neurotrauma, including traumatic brain injury, Alzheimers disease, Huntington's disease, Niemann-Pick disease, Parkinson's disease, motor neuron disease, Amyotrophic Lateral Sclerosis, Sedaghatian-type spondylo-metaphyseal dysplasia, cancer, including breast cancer, kidney injuries including ischemia-reperfusion and oxalic acid-induced kidney damage, rhabdomyolosis and acute renal failure, mitochondrial dysfunction leading to osteoporosis, lysosomal storage diseases including cystinosis, Danon, Fabry, Krabbe, Gaucher, Niemann-Pick, Pompe and Salla diseases.

### CROSS-REFERENCE TO RELATED APPLICATIONS

This application claims priority to U.S. Provisional Application Ser. No. 63/164,843 filed Mar. 23, 2021 and U.S. Provisional Application Ser. No. 63/226,572 filed Jul. 28, 2021, the entire contents of which are incorporated herein by reference.

### STATEMENT OF FEDERALLY FUNDED RESEARCH

Not applicable.

### TECHNICAL FIELD OF THE INVENTION

The present invention relates in general to the field of compositions and methods for using N-acetylcysteine amide (NACA) or (2R,2R′)-3,3′-disulfanediyl bis(2-acetamidopropanamide) (diNACA) for the treatment of diseases and disorders caused or contributed to by ferroptosis.

### BACKGROUND OF THE INVENTION

All eukaryotes require iron. Replication, detoxification, and a cancer-protective form of regulated cell death termed ferroptosis, all depend on iron metabolism. Because excess age-related iron elevation in somatic tissue, particularly in brain, is thought to contribute to degenerative disease, post-developmental interventions to limit ferroptosis may promote healthy aging (Jenkins et al., 2020). Ferroptosis causes or contributes to numerous diseases, disorders and conditions including aging associated with declining cognition, cognitive impairment, declining physical function, physical decline, elevated inflammation, endothelial dysfunction, insulin resistance, central obesity, loss of muscle mass (sarcopenia) (Kumar et al., 2021), neurological diseases and disorders, including neurodegeneration, stroke (Southon et al., 2019), including ischemic stroke (Ren et al., 2020) and post-hemorrhagic stroke damage (Ren et al., 2020; Han et al., 2020) and neurotrauma, including traumatic brain injury (TBI) (Ren et al., 2020), Alzheimers disease (AD) (Ren et al., 2020; Masaldan et al., 2019), Huntington's disease (HD) (Han et al., 2020; Masaldan et al., 2019; Stockwell et al., 2017; Wright et al., 2015), Parkinson's disease (PD) (Han et al., 2020; Masaldan et al., 2019), motor neuron disease (MND) (Masaldan et al., 2019), Amyotrophic Lateral Sclerosis (ALS) (Masaldan et al., 2019), Sedaghatian-type spondylo-metaphyseal dysplasia (SSMD; GPX4) (Fedida et al., 2020), cancer, including breast cancer (Han et al., 2020), kidney injuries including ischemia-reperfusion and oxalic acid-induced kidney damage, rhabdomyolosis and acute renal failure (Han et al., 2020), mitochondrial dysfunction (Che et al., 2021) leading to osteoporosis (Che et al., 2021), lysosomal storage diseases (LSD) including cystinosis, Danon Fabry, Krabbe, Gaucher, Niemann-Pick (Fu et al., 2013), Pompe and Salla diseases (Pierzynowska et al., 2021).

Ferroptosis is a form of regulated cell death, distinct from apoptosis, necroptosis, and other forms of cell death. Ferroptosis is induced by disruption of glutathione (GSH) synthesis or inhibition of glutathione peroxidase 4 (GPX4), exacerbated by iron (Abdalkader et al., 2018). Perturbations in iron homeostasis and iron accumulation feature in numerous neurodegenerative disorders (Masaldan et al., 2019). Proteins such as α-synuclein, tau and amyloid precursor protein that are pathologically associated with neurodegeneration are involved in molecular crosstalk with iron homeostatic proteins. Despite these strong links between altered iron homeostasis and neurodegeneration, the molecular biology to describe the association between enhanced iron levels and neuron death, synaptic impairment and cognitive decline is ill-defined. While lipid peroxidation plays a central role in the execution of ferroptosis, the removal of iron through chelation or genetic modifications appears to extinguish the ferroptotic pathway. Conversely, tissues that harbor elevated iron may be predisposed to ferroptotic damage. These emerging findings are of relevance to neurodegeneration where ferroptotic signaling may offer new targets to mitigate cell death and dysfunction (Masaldan et al., 2019).

Iron-dependent lipid peroxidation is involved in AD, ALS, PD, stroke (Southon et al, 2019), Huntington's disease (Stockwell et al., 2017) and lysosomal storage diseases including cystinosis, Danon, Fabry, Krabbe, Gaucher, Niemann-Pick, Pompe and Salla diseases (Pierzynowska et al., 2021). Iron levels are elevated in affected brain regions in numerous neurological disorders, as are lipid peroxidation products characteristic of ferroptosis. The most potent known inhibitors of ferroptosis are the radical trapping antioxidants (RTAs), liproxstatin-1 and ferrostatin-1 (Dixon et al., 2012), which inhibit ferroptosis with nanomolar efficacy. Ferrostatin-1 and liproxstatin-1 have been reported to rescue ischemia reperfusion injury and stroke. Edaravone, an RTA approved for the treatment of acute ischemic stroke and ALS, was recently reported to have anti-ferroptotic properties (Southon et al., 2019). Other small molecule agents have been found to have anti-ferroptotic activity, e.g., diacetyl-bis(4-methyl-3-thiosemicarbazonato)copperII (CuII(atsm)) ameliorates neurodegeneration and delays disease progression in mouse models of ALS and PD, though the mechanism of action remains uncertain (Southon et al, 2019).

Motor Neuron Disease (MND) is a relentlessly progressive neurodegenerative condition with no effective disease-modifying therapy. MND affects over 450,000 people worldwide (Collaborators et al., 2016) and average survival post diagnosis is only 2-4 years (Marin et al., 2016). Iron dyshomeostasis is a common feature of this disorder (Wang et al., 2020; Yu et al., 2018) with iron and glutathione peroxidase activity long known to be elevated in motor neuron disease (Ince et al., 1994). Together, these results suggest ferroptosis, an iron-dependent form of regulated cell death, as a probable mechanism in the progression of MND (Spasic et al., 2020; Masaldan et al., 2018; Devos et al., 2019). Restoration of total intracellular GSH levels is known to limit ferroptosis and therefore represents a promising therapeutic target for MND. MND involves oxidative stress-associated cell death. Reactive oxygen species (ROS), such as superoxide, are produced during normal cellular processes and can also be created by exogenous toxins and environmental stressors. Superoxide dismutase type 1 (SOD1) is a major cellular antioxidant, and catalyzes the dismutation of superoxide into hydrogen peroxide and water (Pansarasa et al., 2018). Hydrogen peroxide can also be enzymatically eliminated, e.g., by Glutathione Peroxidases, such as GPX4 (Stockwell et al., 2017). Increased oxidative damage to proteins and cell membrane lipids occurs in MND (Simpson et al., 2004; Tohgi et al., 1999) and these observations have focused some therapeutic efforts towards augmenting cellular capacity to buffer against oxidative cell damage. Recent work has implicated ferroptosis (Stockwell et al., 2017), a regulated cell death pathway characterized by iron-dependent lipid peroxidation, in MND pathogenesis (Masaldan et al., 2018). Iron levels are elevated in affected brain regions in MND (Ayton et al., 2015; Grloez et al., 2016), as are lipid peroxidation products characteristic of ferroptosis (Simpson et al., 2004; Tohgi et al., 1999). Experimentally, ferroptosis can be induced by inhibiting GPX4 with the small molecule RSL3 (Stockwell et al., 2017). Ferrostatin-1 and liproxstatin-1, are the prototypical ferroptosis inhibitors, acting as lipid radical quenching agents (Zilka et al., 2017). Despite metabolic instability and relatively poor blood-brain barrier (BBB) penetration (Magtanong et al., 2018), these compounds have been reported to rescue mouse models of Parkinson's disease (do Van et al., 2016). Edaravone, a radical-quenching agent approved for the treatment of MND, was also reported to have anti-ferroptotic properties (Homma et al., 2019).

An ultra-rare disease involving ferroptosis is Sedaghatian-type spondylo-metaphyseal dysplasia (SSMD), a lethal autosomal recessive disorder, featuring skeletal dysplasia, cardiac arrhythmia, and brain anomalies. Accumulation of lipid peroxides causes membrane damage and cell death. Glutathione peroxidase 4 (GPX4) acts as a hydroperoxidase which prevents accumulation of toxic oxidized lipids and blocks ferroptosis, an iron-dependent, non-apoptotic mode of cell death. GPX4 deficiency causes SSMD (Fedida et al., 2020). There are no treatments available as of today. Antioxidants such as Vitamin E, N-acetylcysteine (NAC), Co-Enzyme Q10 have been shown to be effective in experiments, but none of those cross the blood brain barrier (www.curegpx4.org/about-ssmd).

Antioxidants, alpha-tocopherol, butylated hydroxytoluene (BHT) and NAC (Dixon et al., 2012), can block ferroptosis by inhibiting the lipid peroxidation pathway (Han et al., 2020). Other inhibitors of ferroptosis include baicalein (at low concentrations), curcumin (at low concentrations), ferrostatin-1 (Abdalkader et al., 2018; Dixon et al., 2012), liproxstatin-1 (lip 1) Dixon et al., 2012), SRS 11-92, SRS 16-86, beta-carotene, ammonium chloride, PepA-Me, deferoxamine mesylate, 2,2′-bipyridyl, ciclopirox olamine, zileuton, NDGA, PD146176, CDC, AA-861, BW A4C, XJB-5-131, cycloheximide, diarylamine, phenoxazine, tetrahydronaphthyridinols, PMC, TEMPO, rosiglitazone, pioglitazone and troglitazone (Han et al., 2020). The synthesis of tripeptide GSH appears to protect cells from ferroptotic death. The functional activity of GPX4 is dependent on the biosynthesis of GSH (Yang et al., 2014). More specifically, depletion of GSH causes GPX4 inactivation and increases intracellular lipid peroxidation, resulting in ferroptosis (Han et al., 2020).

### NAC and its Derivatives as Candidate Therapeutics for MND

NAC is a clinically widely used thiol antioxidant that functions as a scavenger of free radicals and facilitates the production of the intracellular antioxidant glutathione GSH by reducing extracellular cystine to cysteine. However, attempts to bolster GSH levels in vivo via administration of the GSH precursor, NAC, has yielded mixed results. Furthermore, NAC is not an ideal drug for development due to high hydrophilicity and relatively short biological half-life (Sunitha et al., 2013).

Despite these developments, a need remains for treatment for ferroptosis, and diseases or conditions associated therewith.

### SUMMARY OF THE INVENTION

In one embodiment, the present invention includes a method of treating or alleviating a disease, disorder, or condition associated with ferroptosis in a human subject that comprises: administering to the human patient a therapeutically effective amount of N-acetylcysteine amide (NACA) or (2R,2R′)-3,3′-disulfanediyl bis(2-acetamidopropanamide) (diNACA) sufficient to reduce the symptoms or treat the ferroptosis disease, disorder, or condition. In one aspect, the NACA or diNACA is provided in or with a pharmaceutically acceptable carrier. In another aspect, the NACA or diNACA is administered dermally, orally, intravenously, intramuscularly, enterally, parenterally, topically, sublingually, rectally, or by inhalation, implant, or insert. In another aspect, the NACA or diNACA is administered in daily doses of about 0.01, 0.1, 1, 5, 10, 20, 25, 30, 40, 50, 60, 70, 75, 80, 90, 100, 150, 150, 200, 250, 300, 333, 350, 400, 450, or 500 mg/Kg. In another aspect, the NACA or diNACA is administered two or three times daily. In another aspect, the NACA or diNACA is administered with a second active agent. In another aspect, the NACA or diNACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, propyl gallate, α-tocopherol, citric acid, ethylenediamine tetraacetic acid (EDTA), sorbitol, tartaric acid, or phosphoric acid. In another aspect, the dose for administration is 0.01, 0.1, 1, 10, 100, 150, 150, 300, 333, 400, 500, 600, 700, 750, 800, 900, 1,000, 2,500, 5,000, 7,500, or 10,000 mg per dose. In another aspect, the NACA or diNACA is delivered orally via a mini-tablet, capsule, tablet, effervescent, dual release, mixed release, sachet, powder, or liquid. In another aspect, the therapeutically effective amount decreases a loss of cognition or any physical ability by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, selected from at least one of 2 weeks, one month, 2 months, 3 months, 6 months, one year, 2 years, or 5 years. In another aspect, the disease, disorder, or condition associated with ferroptosis is declining cognition, cognitive impairment, declining physical function, physical decline, elevated inflammation, endothelial dysfunction, insulin resistance, central obesity, or loss of muscle mass (sarcopenia). In another aspect, the disease, disorder, or condition associated with ferroptosis is neurological diseases and disorders, neurodegeneration, stroke, including ischemic stroke and post-hemorrhagic stroke damage and neurotrauma, including traumatic brain injury (TBI). In another aspect, the disease, disorder, or condition associated with ferroptosis is Alzheimer's disease Huntington's disease, Niemann-Pick disease, Parkinson's disease, motor neuron disease, or Sedaghatian-type spondylo-metaphyseal dysplasia. In another aspect, the disease, disorder, or condition associated with ferroptosis is Amyotrophic Lateral Sclerosis (ALS). In another aspect, the disease, disorder, or condition associated with ferroptosis is cancer, a breast cancer, a pancreatic cancer, colorectal cancer, lung cancer, liver cancer, glioma, ovarian cancer, neuroblastoma, head and neck cancer, melanoma, or esophageal cancer. In another aspect, the disease, disorder, or condition associated with ferroptosis is a kidney injury selected from ischemia-reperfusion and oxalic acid-induced kidney damage, rhabdomyolysis, or acute renal failure. In another aspect, the disorder or condition associated with ferroptosis is a mitochondrial dysfunction, osteoporosis, or a lysosomal storage disease. In another aspect, the disease, disorder, or condition associated with ferroptosis is cystinosis, Danon, Fabry, Krabbe, Gaucher, Niemann-Pick, Pompe, or Salla disease.

In another embodiment, the present invention includes a method of treating or alleviating a disease, disorder, or condition associated with ferroptosis in a human subject that comprises: identifying a human patient in need of treatment for ferroptosis or the disorder or condition associated with ferroptosis; and administering to the human patient a therapeutically effective amount of N-acetylcysteine amide (NACA) or (2R,2R′)-3,3′-disulfanediyl bis(2-acetamidopropanamide) (diNACA) sufficient to reduce the symptoms or treat the ferroptosis disease, disorder or condition. In one aspect, the NACA or diNACA is provided in or with a pharmaceutically acceptable carrier. In another aspect, the NACA or diNACA is administered dermally, orally, intravenously, intramuscularly, enterally, parenterally, topically, sublingually, rectally, or by inhalation, implant or insert. In another aspect, the NACA or diNACA is administered in daily doses of about 0.1 to 150 mg/Kg. In another aspect, the NACA or diNACA is administered in daily doses of about 151 to 500 mg/Kg. In another aspect, the NACA or diNACA is administered two or three times daily. In another aspect, the NACA or diNACA is administered with a second active agent. In another aspect, the NACA or diNACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, propyl gallate, α-tocopherol, citric acid, ethylenediamine tetraacetic acid (EDTA), sorbitol, tartaric acid, or phosphoric acid. In another aspect, the dose for administration is about 0.01, 0.1, 1, 5, 10, 20, 25, 30, 40, 50, 60, 70, 75, 80, 90, 100, 150, 150, 200, 250, 300, 333, 350, 400, 450, 500, 600, 700, 750, 800, 900, 1,000, 2,500, 5,000, 7,500, or 10,000 mg per dose. In another aspect, the NACA or diNACA is delivered orally via a mini-tablet, capsule, tablet, effervescent, dual release, mixed release, sachet, powder, or liquid. In another aspect, the therapeutically effective amount preferably refers to the amount of a therapeutic agent that decreases the loss of cognition or any physical ability by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, selected from at least one of 2 weeks, one month, 2 months, 3 months, 6 months, one year, 2 years, or 5 years. In another aspect, the disease, disorder, or condition associated with ferroptosis is declining cognition, cognitive impairment, declining physical function, physical decline, elevated inflammation, endothelial dysfunction, insulin resistance, central obesity, or loss of muscle mass (sarcopenia). In another aspect, the disorder or condition associated with ferroptosis is neurological diseases and disorders, neurodegeneration, stroke, including ischemic stroke and post-hemorrhagic stroke damage and neurotrauma, including traumatic brain injury (TBI). In another aspect, the disorder or condition associated with ferroptosis is Alzheimer's disease Huntington's disease, Niemann-Pick disease, Parkinson's disease, motor neuron disease, or Sedaghatian-type spondylo-metaphyseal dysplasia. In another aspect, the disorder or condition associated with ferroptosis is Amyotrophic Lateral Sclerosis (ALS). In another aspect, the disorder or condition associated with ferroptosis is cancer, a breast cancer, a pancreatic cancer, colorectal cancer, lung cancer, liver cancer, glioma, ovarian cancer, neuroblastoma, head and neck cancer, melanoma, or esophageal cancer. In another aspect, the disorder or condition associated with ferroptosis is kidney injuries selected from ischemia-reperfusion and oxalic acid-induced kidney damage, rhabdomyolysis, or acute renal failure. In another aspect, the disorder or condition associated with ferroptosis is a mitochondrial dysfunction, osteoporosis, or a lysosomal storage disease. In another aspect, the disorder or condition associated with ferroptosis is cystinosis, Danon Fabry, Krabbe Gaucher Niemann-Pick, Pompe, or Salla disease.

In another embodiment, the present invention includes a method of treating or alleviating a disease, disorder, or condition associated with ferroptosis comprising: identifying a human in need of treatment for the disorder or condition associated with ferroptosis; and administering to the human a therapeutically effective amount of N-acetylcysteine amide (NACA) or (2R,2R′)-3,3′-disulfanediyl bis(2-acetamidopropanamide) (diNACA) sufficient to treat the disorder or condition associated with ferroptosis, aging associated with declining cognition, cognitive impairment, declining physical function, physical decline, elevated inflammation, endothelial dysfunction, insulin resistance, central obesity, loss of muscle mass (sarcopenia), neurological diseases and disorders, including neurodegeneration, stroke, including ischemic stroke and post-hemorrhagic stroke damage, and neurotrauma, including traumatic brain injury, Alzheimer's disease, Huntington's disease, Niemann-Pick disease, Parkinson's disease, motor neuron disease, Amyotrophic Lateral Sclerosis, Sedaghatian-type spondylo-metaphyseal dysplasia, cancer, a breast cancer, a pancreatic cancer, colorectal cancer, lung cancer, liver cancer, glioma, ovarian cancer, neuroblastoma, head and neck cancer, melanoma, or esophageal cancer, kidney injuries including ischemia-reperfusion and oxalic acid-induced kidney damage, rhabdomyolysis and acute renal failure, mitochondrial dysfunction leading to osteoporosis, lysosomal storage diseases including cystinosis, Danon Fabry, Krabbe Gaucher Niemann-Pick, Pompe and Salla disease.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C show that NAC and NACA prevent ferroptosis, in vitro. Viability of cells treated with erastin (FIG. 1A, 2 μM), RSL3 (FIG. 1B, 100 nM) or glutamate (FIG. 1C, 100 mM)±liproxstatin-1 (4-250 nM), NAC, NACA or diNACA (8-500 μM) for 24 hours. diNACA solubilized in NMP. Data are means±SEM, n=3.

FIG. 2 shows NMP solvent is toxic to N27 cells. Viability of cells treated with water, dimethyl sulfoxide (DMSO) or NMP (0.3-20% v/v) for 24 hours. Data are means±SEM, n=3.

FIG. 3 shows diNACA partially prevents ferroptosis, in vitro. Viability of cells treated with erastin (2 μM), RSL3 (100 nM) or glutamate (100 mM)±diNACA (2-75 mM) for 24 hours. diNACA solubilized in media. Data are means±SEM, n=3.

FIGS. 4A to 4C show NAC and NACA are similarly effective at preventing ferroptosis, in vitro. Viability of cells treated with erastin (FIG. 4A, 0.1-8 μM), RSL3 (FIG. 4B, 6-400 nM) or glutamate (FIG. 4C, 6-400 mM)±NAC (50 μM), or NACA (50 μM) for 24 hr. NAC and NACA solubilized in media. Data are means±SEM, n=3.

FIGS. 5A to 5C show NAC and NACA are similarly effective at preventing ferroptosis, in vitro. Viability of cells treated with erastin (FIG. 5A, 0.1-4 μM), RSL3 (FIG. 5B, 3-200 nM) or glutamate (FIG. 5C, 3-200 mM)±NAC (50 μM) or NACA (50 μM) for 24 hr. NAC and NACA solubilized in PBS. Data are means±SEM, n=3.

FIGS. 6A to 6F show that pre-treating cells with NAC, NACA or diNACA prior to ferroptosis induction. Viability of cells treated with NAC, NACA or diNACA (FIGS. 6A-6C, 50 μM; FIGS. 6D-6F, 500 μM) for 24 hours and then treated±erastin (FIG. 6A, FIG. 6D 0.1-4 μM), RSL3 (FIG. 6B, FIG. 6E 3-200 nM) or glutamate (FIG. 6C, FIG. 6F 2-100 mM) for an additional 24 hours. NAC, NACA or diNACA were solubilized in PBS. Data are means±SEM, n=3.

FIGS. 7A to 7F show that treating cells with NAC or NACA after ferroptosis induction. Viability of cells treated with erastin (FIG. 7A, FIG. 7D 0.1-4 μM), RSL3 (FIG. 7B, FIG. 7E 3-200 nM) or glutamate (FIG. 7C, FIG. 7F 2-100 mM) for 5 hours and then treated±NAC, NACA or diNACA (FIG. 7A-7C, 50 μM; FIG. 7D-7F, 500 μM) for an additional 19 hours. NAC, NACA or diNACA were solubilized in PBS. Data are means±SEM, n=3.

FIG. 8 shows Caenorhabditis elegans (C. elegans) treatment from the late L4 larval/young adult stage of development and survival curves with 1 or 10 mM NAC, NACA or diNACA versus control.

FIG. 9 shows effects of 1 and 10 mM NAC, NACA and diNACA on C. elegans treated with 10 mM DEM.

FIG. 10 shows rat plasma levels of NAC, NACA and diNACA after oral administration of either 200 mg/kg (mpk) NACA or diNACA.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention relates in general to the field of compositions and methods for using N-acetylcysteine amide (NACA) or (2R,2R′)-3,3′-disulfanediyl bis(2-acetamidopropanamide) (diNACA) for the treatment of diseases, disorders, conditions or abnormalities caused or contributed to by ferroptosis, including aging associated with declining cognition, cognitive impairment, declining physical function, physical decline, elevated inflammation, endothelial dysfunction, insulin resistance, central obesity, loss of muscle mass (sarcopenia), neurological diseases and disorders, including neurodegeneration, stroke, including ischemic stroke and post-hemorrhagic stroke damage, and neurotrauma, including traumatic brain injury, Alzheimer's disease, Huntington's disease, Niemann-Pick disease, Parkinson's disease, motor neuron disease, Amyotrophic Lateral Sclerosis, Sedaghatian-type spondylo-metaphyseal dysplasia, cancer, including breast cancer, kidney injuries including ischemia-reperfusion and oxalic acid-induced kidney damage, rhabdomyolosis and acute renal failure, mitochondrial dysfunction leading to osteoporosis, lysosomal storage diseases including cystinosis, Danon, Fabry, Krabbe, Gaucher, Niemann-Pick, Pompe and Salla diseases.

NACA (NPI-001) is a small molecule antioxidant being developed for treatment of ocular conditions of oxidative stress by Nacuity Pharmaceuticals, PTY, LTD (Safety and Efficacy of NPI-001 Tablets for RP Associated with Usher Syndrome (SLO RP) (ClinicalTrials.gov Identifier: NCT04355689)). An analog, (2R,2R′)-3,3′-disulfanediyl bis(2-acetamidopropanamide) (diNACA) (NPI-002), is another antioxidant drug which serves as a prodrug that is metabolized to other active antioxidants, NACA and NAC (PCT/US21/14819), both of which are antioxidants (Sunitha et al., 2013).

NACA and diNACA have been demonstrated to exhibit antiferroptotic activity (Examples 1-3). NACA potently prevents lipid peroxidation and ferroptosis in cell culture models. diNACA prevents ferroptosis and significantly increases C. elegans lifespan assay in vivo (Examples 1-3).

N-acetyl-L-cysteine amide (NACA), also known as (R)-2-(acetylamino)-3-mercapto-propanamide, N-acetyl-L-cysteinamide, or acetylcysteinamide, has the structure:

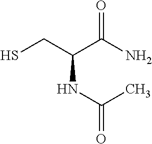

(2R,2R′)-3,3′-disulfanediyl bis(2-acetamidopropanamide) (diNACA), has the structure:

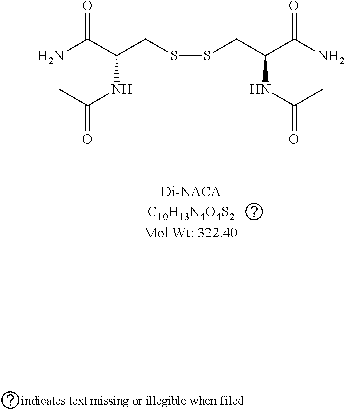 [SMILES: CC(=O)N[C@@H](CSSC[C@H](NC(C)=O)C(N)=O)C(N)=O ; "C10H13N4O4S2"]

(2R,2R′)-3,3′-disulfanediyl bis(2-acetamidopropanamide) (diNACA), the dimer form of N-acetyl-L-cysteineamide, acts as a carrier of NAC or cysteine.

### Caenorhabditis elegans as a Model Organism to Study Ferroptosis Activity

Ferrous iron accumulates over adult lifetime in Caenorhabditis elegans (C. elegans). Glutathione depletion is coupled to ferrous iron elevation in these animals, and both occur in late life to prime cells for ferroptosis. Blocking ferroptosis, either by inhibition of lipid peroxidation or by limiting iron retention, mitigates age-related cell death and markedly increases lifespan and healthspan. Temporal scaling of lifespan is not evident when ferroptosis is inhibited, consistent with this cell death process acting at specific life phases to induce organismal frailty, rather than contributing to a constant aging rate (Jenkins et al., 2020).

The Drug Age Database of Anti-Aging Drugs contains an extensive compilation of drugs, compounds, and supplements (including natural products and nutraceuticals) with anti-aging properties that extend longevity in model organisms (genomics.senescence.info/drugs/). At the time of writing this application, studies of effects of 970 agents on the longevity of C. elegans have been reported. For example, the four most potent life extension agents for C. elegans listed are thioflavin T (78%, the most potent agent in the database), bacitracin (74%), acetaminophen (66%) and NAC, 64% (genomics.senescence.info/drugs/).

In vitro studies showed good antiferroptotic activity of NAC, NACA and poor activity for diNACA. However, surprisingly, in vivo studies showed excellent antiferroptotic activity of diNACA, in fact, greater than 970 cataloged compounds that have been tested in the C. elegans model of ferroptosis (genomics.senescence.info/drugs/).

NAC and NACA were able to dose-dependently prevent cell death induced by either erastin, RSL3 or glutamate in cultured neurons, demonstrating these compounds are anti-ferroptotic. NAC and NACA were effective at micromolar concentrations in vitro and therefore not as effective as the canonical anti-ferroptotic compound Lip-1, which is effective at nanomolar levels. NAC and NACA were similarly effective at preventing ferroptosis under a range of different concentrations. NACA did not outperform NAC when concentrations of ferroptosis inducers or time of ferroptosis induction were altered. Nevertheless, NACA may be more effective in vivo, given it is more bioavailable (yields nearly double [NAC] compared to NAC alone) than NAC (He et al., 2020).

diNACA in vitro partially prevented cell death induced by erastin, glutamate and to a lesser extent RSL3, indicating diNACA has some anti-ferroptotic properties. diNACA was only effective in vitro when used at millimolar concentrations.

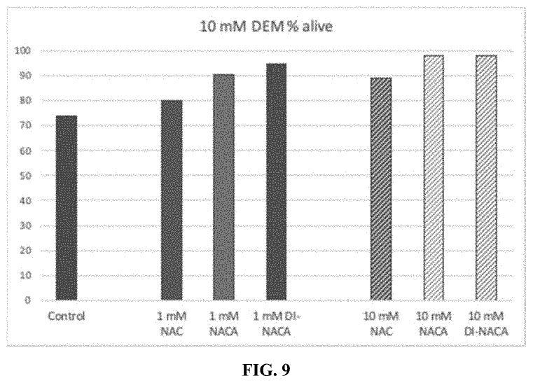

Surprisingly, in vivo screening showed excellent antiferroptic activity of diNACA, over NAC and NACA. The C. elegans Lifespan assay has been established as a valid screening assay for anti-ferroptotic activity (Jenkins et al., 2020). Using C. elegans Lifespan assay, oral dosing of NAC, NACA and diNACA was conducted. Surprisingly, marked increase in lifespan was observed with 10 mM NACA, 1 mM and 10 mM diNACA, with a minor increase observed for with 10 mM NAC and 1 mM NACA (Tables 3 and 4; FIGS. 8, 9, 10 and 11). Studies of the effects of 970 agents on longevity of C. elegans have been reported. Of these, the four most potent life extension agents for C. elegans listed are thioflavin T (78%, the most potent agent in the database), bacitracin (74%), acetaminophen (66%) and NAC, 64% (genomics.senescence.info/drugs/). Hence, surprisingly, diNACA exhibited mediocre in vitro anti-ferroptotic activity but excellent in vivo (C. elegans) anti-ferroptotic activity. The percentage lifespan extension observed with diNACA was a 125% increase in median lifespan.

GSH is a tripeptide, c-L-glutamyl-L-cysteinyl-glycine, found in all mammalian tissues. It has several important functions including detoxification of electrophiles, scavenging ROS, maintaining the thiol status of proteins, and regeneration of the reduced forms of vitamins C and E. GSH is the dominant non-protein thiol in mammalian cells; as such it is essential in maintaining the intracellular redox balance and the essential thiol status of proteins. Also, it is necessary for the function of some antioxidant enzymes such as the glutathione peroxidases.

Intracellular GSH levels are determined by the balance between production and loss. Production results from de novo synthesis and regeneration of GSH from GSSG by GSSG reductase. Generally, there is sufficient capacity in the GSSG reductase system to maintain all intracellular GSH in the reduced state, so little can be gained by ramping up that pathway. The major source of loss of intracellular GSH is transport out of cells. Intracellular GSH levels range from 1-8 mM while extracellular levels are only a few μM; this large concentration gradient essentially precludes transport of GSH into cells and once it is transported out of cells, it is rapidly degraded by γ-glutamyltranspeptidase. Inhibition of GSH transporters could theoretically increase intracellular GSH levels but is potentially problematic because the transporters are not specific for GSH, and their suppression could lead imbalance of other amino acids and peptides. Thus, intracellular GSH levels are modulated primarily by changes in synthesis.

GSH is synthesized in the cytosol of virtually all cells by two ATP-requiring enzymatic steps: L-glutamate+L-cysteine+ATP[→]γ-glutamyl-L-cysteine+ADP+Pi and γ-glutamyl-L-cysteine+L-glycine+ATP[→]GSH+ADP+Pi. The first reaction is rate-limiting and is catalyzed by glutamate cysteine ligase (GCL, EC 6.3.2.2). GCL is composed of a 73 Kd heavy catalytic subunit (GCLC) and a 30 Kd modifier subunit (GCLM), which are encoded by different genes. GCCL is regulated by non-allosteric competitive inhibition of GSH (Ki=2.3 mM) and by the availability of L-cysteine. The apparent Kₘ of GLC for glutamate is 1.8 mM and intracellular glutamate concentration is roughly 10-fold higher so that glutamate is not limiting, but the Km for cysteine is 0.1-0.3 mM, which approximates its intracellular concentration. The second reaction is catalyzed by GSH synthase (GS, EC 6.3.2.3), which is 118 Kd and composed of two identical subunits. While GS is not felt to be important in regulation of GSH synthesis under normal conditions, it may play a role under stressful conditions because in response to surgical trauma, GSH levels and GS activity were reduced while GCL activity was unchanged. Furthermore, compared to increased expression of GCLC alone, increased expression of both GCLC and GS resulted in higher levels of GSH. To maximize the effects of increasing synthetic enzymes, it is necessary to provide increased levels of cysteine. In cultured neurons, 90% of cysteine uptake occurs through by the sodium-dependent excitatory amino acid transporter (EAAT) system. There are five EAATs and cysteine uptake by neurons occurs predominantly by EAAT3 more commonly known as excitatory amino acid carrier-1 (EAAC1). Under normal circumstances most EAAC1 is in the ER and only translocate to the plasma membrane when activated. This translocation is negatively regulated by glutamate transporter associated protein 3-18 (GTRAP3-18) and suppression of GTRAP3-18) increased GSH levels in neurons. Thus, internalization of cysteine provides a roadblock for GSH synthesis, but fortunately it can be bypassed by NAC which readily enters cells even in the absence of activated EAAC1. Systemically administered NAC gains access to the CNS, increases GSH levels, and provides benefit in neurodegenerative disorders in which oxidative stress is an important part of the pathogenesis.

NAC is used for the treatment of acetaminophen overdose at a dose of 140 mg/kg as the loading dose, followed by 70 mg/kg every 4 hours for 17 doses, starting 4 hours after the loading dose. In clinical studies, NAC has been administered orally from 400 to 1000 mg once daily and from 200 to 600 mg three times daily. However, following an oral dose of 600 mg in humans, NAC is rapidly absorbed and then rapidly cleared. The plasma half-life of NAC has been reported to be 2.5 hours and no NAC is detectable 10-12 hours after administration. During absorption, NAC is rapidly metabolized to cysteine, which is a direct precursor of glutathione. In accordance with an embodiment, the present invention provides a method for the prevention, amelioration, or treatment of a disease or condition associated with oxidative stress in a subject comprising administration of a therapeutically effective amount of NACA, to increase the amount of glutathione expressed in the tissues of the subject.

### EXAMPLE 1

### Effects of NAC, NACA and diNACA on Ferroptosis In Vitro

Materials and Methods. The objective of this study was screening of NAC, NACA and diNACA to evaluate their anti-ferroptotic activity in cultured neuronal cells. N27 cells, derived from E12 rat mesencephalic tissue (Merck, Australia), were cultured in RPMI 1640 media (ThermoFisher Scientific) supplemented with 10% fetal calf serum (Bovogen, Australia) and penicillin and streptomycin (ThermoFisher Scientific, Australia). Cells were cultured at 37° C. with 5% CO₂. Cellular ferroptosis assays were used to evaluate anti-ferroptotic activity of test articles (Southon et al., 2019). Ferroptosis was induced over 24 hours with either erastin, RSL3 or glutamate at concentrations indicated in figure legends. Liproxstatin-1 (lip1) was used as an anti-ferroptotic control. NAC, NACA and diNACA were added at the same time as ferroptosis inducers unless otherwise stated in figure legends. Cell viability was measured by 3-[4,5-dimethylthiazol-2-yl]-2,5-diphenyltetrazolium bromide (MTT) assay, absorbance measured at 570 nm using a PowerWave XS microplate spectrophotometer (BioTek Instruments, Winooski, USA), and expressed as a percentage of control cells (Southon et al., 2019).

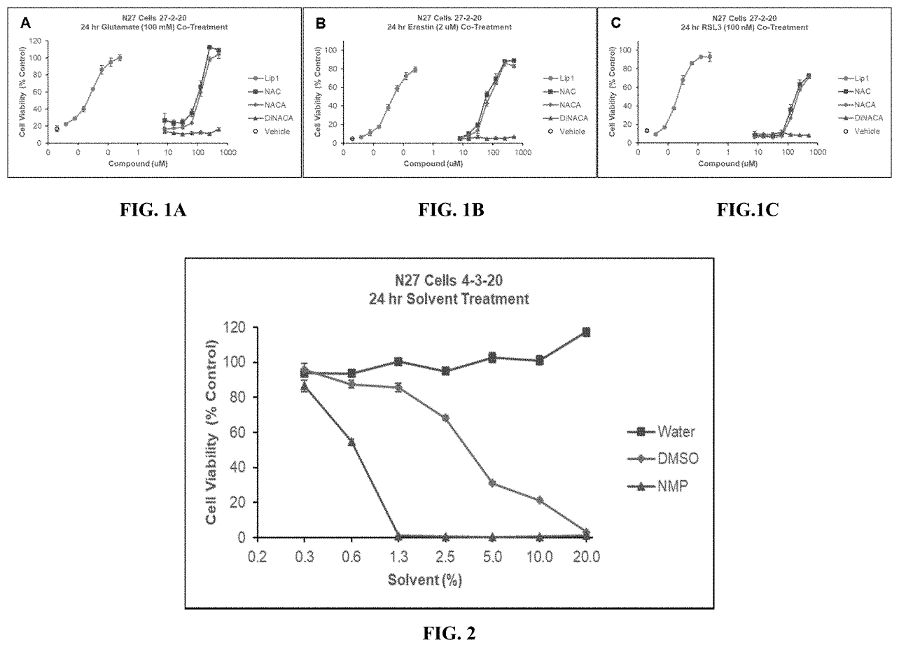

Results. N27 cells were used to evaluate the anti-ferroptotic activity of test compounds (FIGS. 1A, 1B, 1C). NAC and NACA were solubilized in water. diNACA was solubilized in N-methyl pyrrolidone (NMP) as it is not soluble in water or DMSO (Nacuity data on file). Lip1 prevented ferroptosis induced by erastin (FIG. 1A), RSL3 (FIG. 1B) and glutamate (FIG. 1C) with nanomolar efficacy as previously reported (Southon et al., 2019). NAC and NACA prevented ferroptosis induced by erastin, RSL3 and glutamate with efficacy in the micromolar range. diNACA did not prevent ferroptosis induced by erastin, RSL3 or glutamate (FIGS. 1A to 1C). There was suspicion that the NMP solvent may be toxic and masking any potential anti-ferroptotic activity of diNACA.

### Effect of NMP on N27 Cells

The toxicity profile of solvents NMP, DMSO and water were evaluated in N27 cells (FIG. 2). Water did not reduce cell viability at concentrations up to 20% [NAC and NACA were used at a maximum concentration of 500 μM (FIG. 1), which corresponded to 1% water.]. DMSO reduced cell viability at concentrations over 1.3%, which is consistent with unpublished observations. DMSO concentrations in this study did not exceed 1%. NMP reduced cell viability at concentrations over 0.3%. diNACA concentrations of 250 and 500 μM (FIG. 1) correspond to 0.5 and 1% NMP respectively, which are toxic to N27 cells (FIG. 2). diNACA solubilized in NMP should therefore only be used at concentrations up to 125 μM in NHP.

### Effects of diNACA Solubilized in Media on Ferroptosis In Vitro

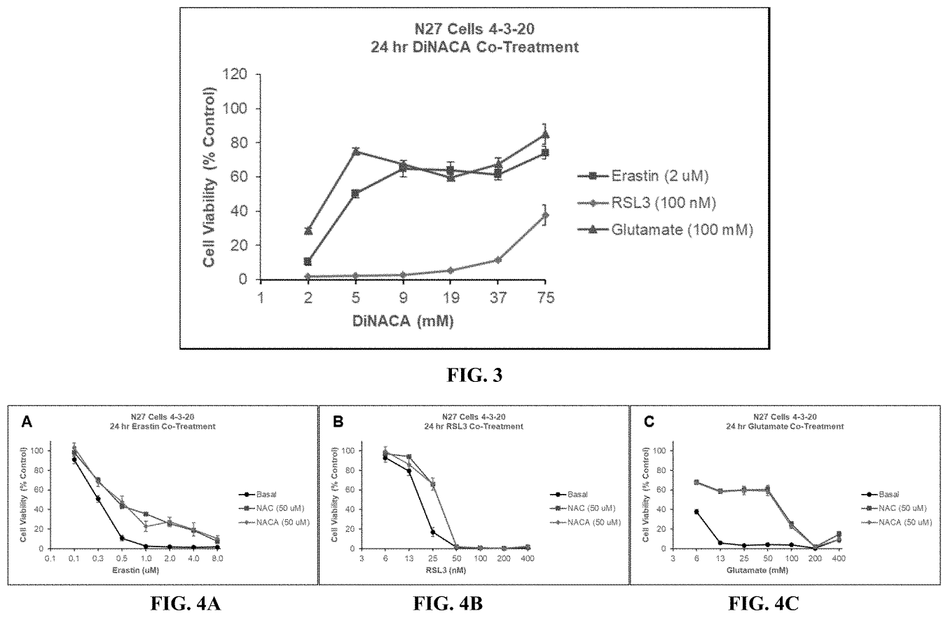

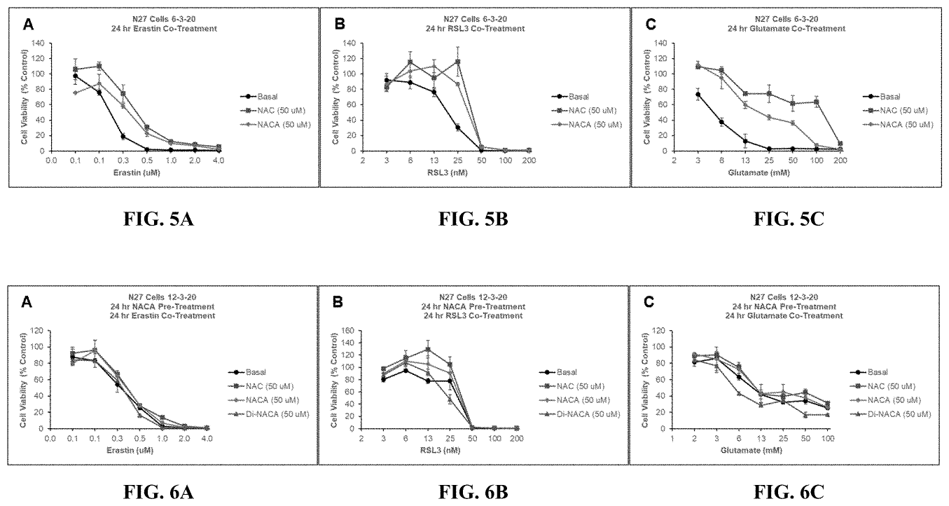

To investigate diNACA concentrations over 125 μM without confounding effects of NMP toxicity, diNACA was added directly to cell culture media and sonicated. The anti-ferroptotic activity of millimolar diNACA concentrations was then evaluated (FIG. 3). diNACA partially prevented erastin and glutamate-induced toxicity when used at concentrations ≥5mM, with little additional benefit seen at concentrations up to 75 mM. diNACA was less effective at preventing RSL3 toxicity with a small effect seen with 75 mM. diNACA was effective at preventing ferroptosis in the millimolar range (FIG. 3), whereas NAC and NACA solubilized in media (FIGS. 4A to 4C) or PBS (FIGS. 5A to 5C) were effective in the micromolar range, in vitro. FIGS. 4A to 4C show NAC and NACA are similarly effective at preventing ferroptosis. Viability of cells treated with erastin (FIG. 4A, 0.1-8 μM), RSL3 (FIG. 4B, 6-400 nM) or glutamate (FIG. 4C, 6-400 mM)±NAC (50 μM) or NACA (50 μM) for 24 hr. NAC and NACA solubilized in media. Data are means±SEM, n=3. FIGS. 5A to 5C show NAC and NACA are similarly effective at preventing ferroptosis. Viability of cells treated with erastin (FIG. 5A, 0.1-4 μM), RSL3 (FIG. 5B, 3-200 nM) or glutamate (FIG. 5C, 3-200 mM)±NAC (50 μM) or NACA (50 μM) for 24 hr. NAC and NACA solubilized in PBS. Data are means±SEM, n=3.

### Effects of NAC, NACA or diNACA Pretreatment on Ferroptosis In Vitro

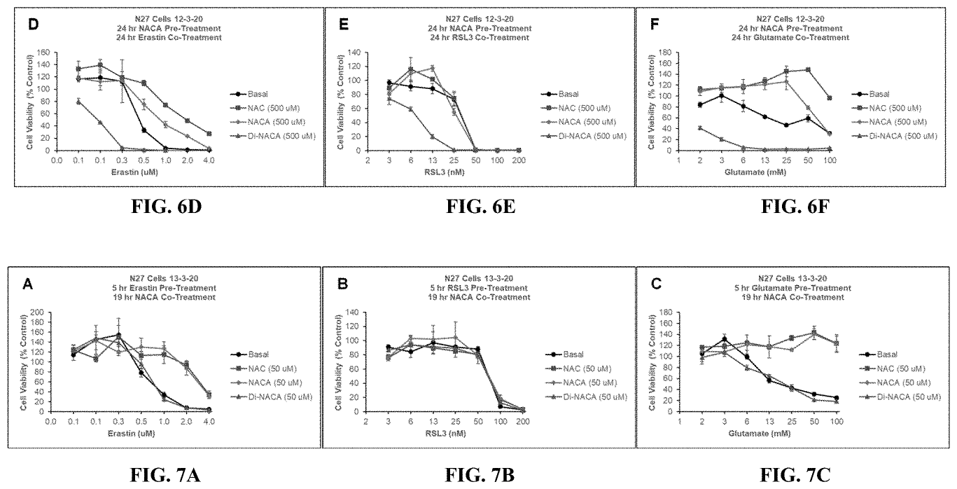

Pretreatment of cells with NAC, NACA or diNACA in PBS prior to ferroptosis induction did not differentiate anti-ferroptotic activity (FIG. 6A to 6F). FIGS. 6A to 6F show that pre-treating cells with NAC, NACA or diNACA prior to ferroptosis induction does not differentiate anti-ferroptotic activity. Viability of cells treated with NAC, NACA or diNACA (FIG. 6A-6C, 50 μM; FIG. 6D-6F, 500 μM) for 24 hours and then treated±erastin (FIGS. 6A, 6D 0.1-4 μM), RSL3 (FIGS. 6B, 6E 3-200 nM) or glutamate (FIGS. 6C, 6F 2-100 mM) for an additional 24 hours. NAC, NACA or diNACA were solubilized in PBS. Data are means±SEM, n=3.

### Effects of NAC, NACA or diNACA Post-Treatment on Ferroptosis In Vitro

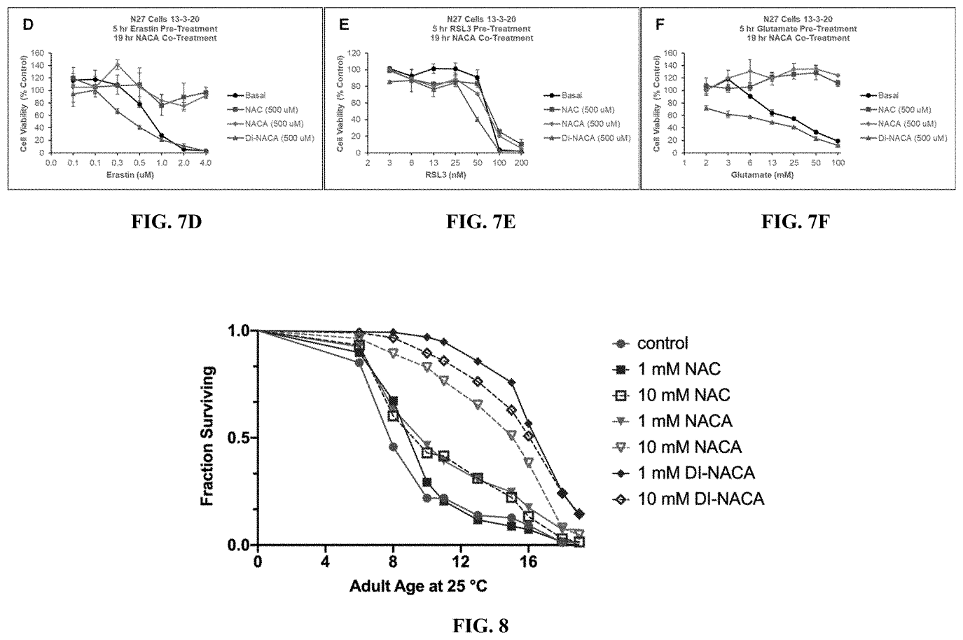

Post-treatment of cells with NAC, NACA or diNACA in PBS prior to ferroptosis induction did not differentiate anti-ferroptotic activity (FIG. 7A to 7F). FIGS. 7A to 7F show that treating cells with NAC or NACA after ferroptosis induction does not differentiate anti-ferroptotic activity. Viability of cells treated with erastin (FIGS. 7A, 7D 0.1-4 μM), RSL3 (FIGS. 7B, 7E 3-200 nM) or glutamate (FIGS. 7C, 7F 2-100 mM) for 5 hours and then treated±NAC, NACA or diNACA (FIG. 7A-7C, 50 μM; FIG. 7D-7F, 500 μM) for an additional 19 hours. NAC, NACA or diNACA were solubilized in PBS. Data are means±SEM, n=3.

### EXAMPLE 2

### Pilot Comparison of NAC vs NACA vs DINACA Using Caenorhabditis elegans Lifespan Assay

Materials and Methods. diNACA was added to neat dimethyl sulfoxide (DMSO; Sigma-Aldrich) then added to the molten Nematode Growth Medium (NGM) at 55° C. to a final concentration of 1% v/v DMSO. diNACA was not fully soluble in DMSO, forming a suspension which dissolved upon addition to the NGM. NAC and NACA were dissolved in sterile water then added to the molten NGM at 55° C. Media containing equivalent vehicle alone (1% v/v DMSO) was used for comparison. 1% v/v DMSO was also added to NAC and NACA plates for consistency, as DMSO itself may affect lifespan. Standard overnight culture of the Escherichia coli strain OP50 was used as the food source. Lifespan data were collected at 25 (±1) ° C. using the temperature sensitive-sterile strain TJ1060 [spe-9(hc88); rrf-3(b26)] as a proxy for wildtype. A developmentally synchronous population was obtained by transferring egg-laying adults to fresh plates at 16° C. for 4 hours. The adults were removed and the plates with eggs then transferred to 25° C. to ensure sterility. After 48 hours at 25° C., when worms were at the late L4 larval/young adult stage, 25-35 nematodes were transferred to fresh plates containing either vehicle control (1% v/v DMSO), 1 mM NAC, 10 mM NAC, 1 mM NACA, 10 mM NACA, 1 mM diNACA, and 10 mM diNACA. All plates were coded to allowing blinding of the experimenter to the treatment regime during scoring. Nematodes were scored for survival at 1 to 2-day intervals and transferred to freshly prepared plates as needed (every 2-4 days). Worms lost not due to death were recorded as censored.

Results. Data was analyzed using GraphPad Prism 9, with survival curves compared using a long-rank (Mantel-Cox) test. P values are given for pair-wise comparisons to the vehicle control population. Surprisingly, diNACA increase survival over NAC and NACA (Table 3). Surprisingly, diNACA marked increase in lifespan was observed with 10 mM NACA, 1 mM and 10 mM diNACA, with a minor increase observed for with 10 mM NAC and 1 mM NACA. Surprisingly, diNACA more than doubled the lifespan of C. elegans (FIG. 8).

TABLE 3
Effects of test articles on survival.

| | Median | | | |
|---|---|---|---|---|
| | survival | # | # | |
| Treatment | (Days) | Deaths | Censored | P value |
| control | 8 | 100 | 6 | |
| 1 mM NAC | 10 | 72 | 15 | 0.2368 |
| 10 mM NAC | 10 | 75 | 12 | 0.0172 |
| 1 mM NACA | 10 | 67 | 8 | 0.0036 |
| 10 mM NACA | 16 | 61 | 17 | <0.0001 |
| 1 mM diNACA | 18 | 113 | 3 | <0.0001 |
| 10 mM diNACA | 18 | 71 | 9 | <0.0001 |

### EXAMPLE 3

### Effects of NAC, NACA and diNACA on Ferroptosis in the Diethyl Maleate Assay

Materials and Methods. Effects of NAC, NACA and diNACA on diethyl maleate (DEM; Sigma-Aldrich)-induced toxicity in C. elegans was evaluated. (10 mM DEM alone conjugates GSH causing 50% depletion of GSH and 50% lethality after 24 hour exposure (Jenkins et al., 2020)). DEM was added to neat DMSO and added to molten NGM at 55° C. to a final concentration of 10 mM DEM and 0.5% v/v DMSO. Plates were seeded with E. coli strain OP50. Data were collected at 25 (±1) ° C. using the temperature sensitive-sterile strain TJ1060. A synchronous population was obtained by transferring egg-laying adults to fresh plates at 16° C. for 2-3 hours. The adults were removed and the plates with eggs then transferred to 25° C. to ensure sterility. After 48 hours at 25° C., when worms were at the late L4/young adult stage, 70 nematodes were transferred to fresh plates containing either vehicle control, 1 mM NAC, 10 mM NAC, 1 mM NACA, 10 mM NACA, 1 mM diNACA, 10 mM diNACA (all with 1.0% v/v DMSO). Worms were aged at 25° C. for a further 4 days and then transferred to DEM plates, 35-45 per plate, two plates per treatment. Survival, determined by touch-provoked movement, was scored at 24 hours after exposure to DEM.

Results. Survival of control was slightly higher than previously observed in the laboratory, perhaps due to higher level of DMSO (1%) in pre-treatment plates. Increased dispersal was noted on 1 mM diNACA plates. Results suggest rescue by 10 mM NAC, and both 1 and 10 mM of NACA and diNACA (Table 4). At 1 mM, NACA and diNACA were superior to NAC regarding survival after 10 mM DEM (FIG. 8).

TABLE 4
Percent survival by test article in DEM assay.

| | Number of | |
|---|---|---|
| | animals | % alive |
| Control | 69 | 74 |
| 1 mM NAC | 66 | 80 |
| 1 mM NACA | 65 | 91 |
| 1 mM diNACA | 40 | 95 |
| 10 mM NAC | 74 | 89 |
| 10 mM NACA | 60 | 98 |
| 10 mM diNACA | 59 | 98 |

### EXAMPLE 4

### Bioavailability of diNACA, NACA and NAC after Oral Administration of diNACA or NACA

Materials and Methods. Bioavailability of diNACA after oral administration in rat was evaluated. NACA and diNACA, separately, were suspended in phosphate-buffered saline (pH 7) and dosed to rats by oral gavage. Blood specimens were collected and processed to produce plasma. Plasma specimens were analyzed for NAC, NACA and diNACA concentration.

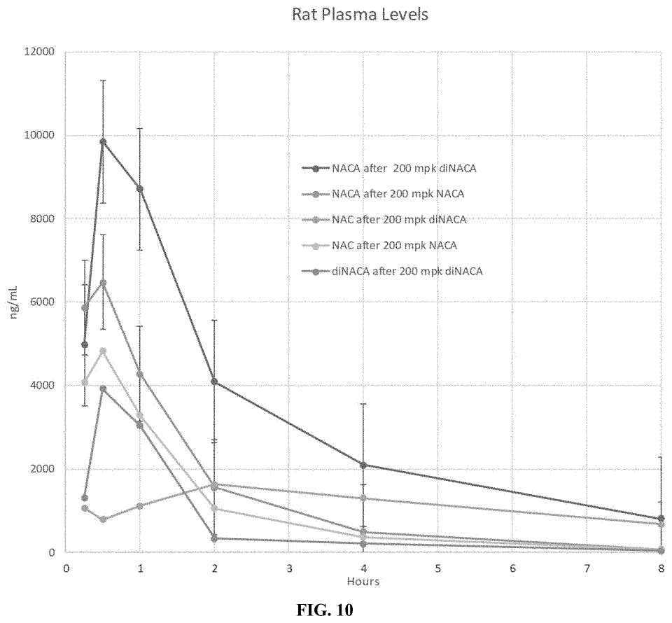

Results. FIG. 10 shows rat plasma levels of NAC, NACA and diNACA after oral administration of either 200 mg/kg (mpk) NACA or diNACA (PCT/US21/14819). Surprisingly, diNACA is well absorbed after oral administration to rats. Also, surprisingly, diNACA is metabolized to NACA and NAC to yield greater levels for longer periods of time compared to oral dosing with NACA.

As used herein, “active oxygen species” or “reactive oxygen species” are understood as transfer of one or two electrons produces superoxide, an anion with the form O₂⁻, or peroxide anions, having the formula O₂²⁻, or compounds containing an O—O single bond, for example hydrogen peroxides and lipid peroxides. Such superoxides and peroxides are highly reactive and can cause damage to cellular components including proteins, nucleic acids, and lipids.

As used herein, the term “agent” refers to a therapeutically active compounds or a potentially therapeutic active compound, e.g., an antioxidant. An agent can be a previously known or unknown compound. As used herein, an agent is typically a non-cell-based compound, however, an agent can include a biological therapeutic agent, e.g., peptide or nucleic acid therapeutic, e.g., siRNA, shRNA, cytokine, antibody, etc.

Oxidation is a chemical reaction that transfers electrons from a substance to an oxidizing agent. Such reactions can be promoted by or produce superoxide anions or peroxides. Oxidation reactions can produce free radicals, which start chain reactions that damage cells. Antioxidants terminate these chain reactions by removing free radical intermediates and inhibit other oxidation reactions by being oxidized themselves. As a result, antioxidants are often reducing agents such as thiols, ascorbic acid, or polyphenols. Antioxidants include, but are not limited to, α-tocopherol, ascorbic acid, Mn (III)tetrakis (4-benzoic acid) porphyrin, α-lipoic acid, and NAC.

As used herein, the terms “effective amount” or “effective doses” refer to that amount of an agent to produce the intended pharmacological, therapeutic, or preventive results. The pharmacologically effective amount results in the amelioration of one or more signs or symptoms of a disease or condition or the advancement of a disease or conditions or causes the regression of the disease or condition. For example, a therapeutically effective amount preferably refers to the amount of a therapeutic agent that decreases disease or condition symptoms by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such as treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater toxicity.

Thus, in connection with the administration of a drug, a drug which is “effective against” a disease or condition indicates that administration in a clinically appropriate manner results in a beneficial effect for at least a statistically significant fraction of patients, such as an improvement of symptoms, a cure, a reduction in disease signs or symptoms, extension of life, improvement in quality of life, or other effect generally recognized as positive by medical doctors familiar with treating the particular type of disease or condition.

As used herein, the terms “peroxidases” or “a peroxide metabolizing enzyme” refer to a large family of enzymes that typically catalyze a reaction of the form:

ROOR₁+electron donor (2 e−)+2H+→ROH+R₁OH

For many of these enzymes the optimal substrate is hydrogen peroxide, wherein each R is H, but others are more active with organic hydroperoxides such as lipid peroxides. Peroxidases can contain a heme cofactor in their active sites, or redox-active cysteine or selenocysteine residues.

As used herein, the term phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers are acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for dermal, oral, intravenous, intramuscular, enteral, parenteral, topical, sublingual, rectal, or by inhalation, implant or insert routes of administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

As used herein, the term “small molecule” refers to a compound, typically an organic compound, having a molecular weight of no more than about 1500 Da, 1000 Da, 750 Da, or 500 Da. In an embodiment, a small molecule does not include a polypeptide or nucleic acid including only natural amino acids and/or nucleotides.

As used herein, the term “subject” refers to living organisms, in particular, humans. In certain embodiments, the living organism is an animal, in certain preferred embodiments, the subject is a mammal, in certain embodiments, the subject is a domesticated mammal or a primate including a non-human primate. Examples of subject include humans, monkeys, dogs, cats, mice, rates, cows, horses, goats, and sheep. A human subject may also be referred to as a subject or patient.

A patient or subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition or syndrome. Methods for identification of subjects suffering from or suspected of suffering from ferroptosis and diseases or conditions associated with the same is within the ability of those in the art. Subjects suffering from, and suspected of suffering from, a specific disease, condition, or syndrome are not necessarily two distinct groups.

As used herein, “superoxide dismutase” is understood as an enzyme that dismutation of superoxide into oxygen and hydrogen peroxide. Examples include, but are not limited to SOD1, SOD2, and SOD3. Sod1 and SOD3 are two isoforms of Cu—Zn-containing superoxide dismutase enzymes that exists in mammals. Cu—Zn-SOD or SOD1, is found in the intracellular space, and extracellular SOD (ECSOD or SOD3) predominantly is found in the extracellular matrix of most tissues.

As used herein, the term “therapeutically effective amount,” refers to an amount of an agent which is effective, upon single or multiple dose administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with a conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1985). Formulations for parenteral administration may contain common excipients such as, e.g., sterile water or saline, polyalkylene glycols such as polyethylene glycol, oils of vegetable origin, hydrogenated naphthalenes, and the like. In particular, biocompatible, biodegradable lactide polymer, lactide/glycolide copolymer, or polyoxyethylene-polyoxypropylene copolymers may be useful excipients to control the release of certain agents.

The thiol moiety of NAC, NACA, or diNACA and their respective internal standards oxidizes quickly in plasma through formation of disulfides. In order to determine total NAC and total NACA levels in plasma, tris(2-carboxyethyl)phosphine (TCEP) is added during the extraction to reduce disulfide bonds. Ammonium bicarbonate is added to control sample pH near neutral, as TCEP will otherwise acidify the aliquoted samples and hinder derivatization. The free analyte is then derivatized to a stable thioether using 2-chloro-1-methylpyridinium iodide (CMPI). A sample volume of 25.0 μL was aliquoted into a 1.2 mL 96-well plate to which was added, in sequence, 25.0 μL internal standard solution (1000 ng/mL NAC-D3 and 1000 ng/mL NACA-D3 in water), 50.0 μL of ammonium bicarbonate (100 mM), 5.0 μL CMPI (60 mM in water), and 5.0 μL of TCEP (60 mM in water). Samples were allowed to react for 30 minutes. To precipitate proteins, 500 μL of acetonitrile was then added to all samples. The plate was covered and the mixtures were shaken and centrifuged. A 50.0 μL aliquot of the supernatant was transferred from each well to a clean plate containing 400 μL of water-acetonitrile (25-75) in each well and mixed well prior to LC-MS injection.

Thioether derivatives of NAC and NACA for LCMS analyses. Samples were analyzed on a Waters Acquity liquid chromatograph interfaced with a Thermo Scientific TSQ Vantage triple quadrupole mass spectrometer with ESI ionization. Each extracted sample was injected (5.0 μL) onto a Waters BEH HILIC column (2.1×100 mm; 1.7 μm) equilibrated at 35° C. Mobile Phase A was ammonium formate (25 mM, pH 3.8). Mobile Phase B was acetonitrile.

TABLE 1
The LC gradient.

| Time | Flow Rate | % MP | % MP |
|---|---|---|---|
| (min) | (mL/min) | A | B |
| 0.00 | 0.500 | 25.0 | 75.0 |
| 2.30 | 0.500 | 25.0 | 75.0 |

The retention time, mass transition and precursor charge state for each compound are as follows.

TABLE 2
shows the masses for the CMPI thioether derivatives.

| | Expected | | Product | Charge |
|---|---|---|---|---|
| | Retention | Precursor | Observed | State of |
| | Time | Mass/Charge | Mass/Charge | Precursor |
| Compound | (min) | (m/z) | (m/z) | Ion |
| N-Acetyl-L-Cysteine (NAC) | 1.90 | 255.080 | 126.16 | +1 |
| N-Acetylcysteine amide (NACA) | 1.25 | 254.096 | 126.16 | +1 |
| N-Acetyl-L-Cys-D₃ | 1.90 | 258.099 | 126.15 | +1 |
| N-Acetyl-L-Cysteine-D₃ | 1.25 | 257.115 | 126.15 | +1 |

Peak area ratios from the calibration standard responses were regressed using a (1/concentration²) linear fit for NAC, NACA or diNACA.

It will be appreciated that the actual preferred amounts of active compounds used in each therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted regarding the forgoing guidelines. Ranges provided herein are understood to be shorthand for all of the values within the range.

As used herein, the embodiments of this invention are defined to include pharmaceutically acceptable derivatives thereof. A “pharmaceutically acceptable derivative” means any pharmaceutically salt, ester, salt of an ester, or other derivative of a compound of this invention which, upon administration to a recipient, is capable of providing (directly or indirectly) a compound of this invention. Particularly favored derivatives are those that increase the bioavailability of the compounds of this invention when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The embodiments of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-napthalenesulfonate, nicotinate, nitrate, palmoate, phosphate, picrate, pivalate, propionate, salicylate, succinate, sulfate, tartrate, tosylate, and undecanoate. Salts derived from appropriate bases include alkali metal (e.g., sodium), alkaline earth metal (e.g., magnesium), ammonium and N-(alkyl)4+ salts. This invention also includes the quaternization of any basic nitrogen-containing groups of the compounds disclosed herein. Water or oil-soluble or dispersible products may be obtained by such quaternization.

The embodiments of the invention can, for example, be administered by injection, intravenously, intraarterially, subdermally, intramuscularly, or subcutaneously; or orally, buccally, nasally, transmucosally, directly to a diseased organ by catheter, topically, or in an ophthalmic preparation, with a dosage ranging from about 0.001 to about 100 mg/kg of body weight, or according to the requirements of the particular drug and more preferably from 0.5-10 mg/kg of body weight of NACA or diNACA. It is understood that when a compound is delivered directly, considerations such as body weight have less bearing on the dose.

Frequency of dosing will depend on the agent administered, the progression of the disease or condition in the subject, and other considerations known to those of skill in the art. For example, pharmacokinetic and pharmacodynamics considerations for compositions delivered systemically. Therefore, dosing can be as infrequent as once a month, once every three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly.

Dosing may be determined in conjunction with monitoring of one or more signs or symptoms of amyotrophic lateral sclerosis (ALS), such as, ⋅muscle twitches in the arm, leg, shoulder, or tongue, loss of motor control in the hands and arms, impairment in the use of the arms and legs, difficulty chewing or swallowing, tripping and falling, dropping things, persistent fatigue, muscle cramps, tight and stiff muscles (spasticity), muscle weakness affecting an arm, a leg, the neck, or diaphragm, slurred and nasal speech, and/or uncontrollable periods of laughing or crying. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity ad course of the disease, condition or symptoms, the patient's disposition to the disease, condition or symptoms and the judgment of the treating physician.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPAN® and/or other similar emulsifying agents or bioavailability enhancers which are commonly used in the manufacture of pharmaceutically acceptable solid, liquid, or other dosage forms may also be used for the purposes of formulation.

In one or more embodiments, NACA or diNACA is administered in daily doses of about 0.1 to 500 mg/Kg, e.g., 0.1, 1, 5, 10, 20, 25, 30, 40, 50, 60, 70, 75, 80, 90, 100, 150, 150, 200, 250, 300, 333, 350, 400, 450, or 500 mg/Kg. In other embodiments, NACA or diNACA is administered two or three times daily. In another aspect, NACA or diNACA is administered with a second active agent selected from ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

In some embodiments, the dose of NACA or diNACA for administration is, 0.1, 1, 5, 10, 20, 25, 30, 40, 50, 60, 70, 75, 80, 90, 100, 150, 150, 200, 250, 300, 333, 350, 400, 450, or 500 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the NACA is delivered orally via a mini-tablet, capsule, tablet, effervescent, dual release, mixed release, sachet, powder, or liquid. In another aspect, the NACA or diNACA is administered prophylactically to prevent ferroptosis and diseases or conditions associated with the same.

In another embodiment, the present invention includes a method for the treatment of Niemann Pick Disease or Huntington's Disease comprising: identifying a human in need of treatment for age-related macular degeneration; and administering to the human a therapeutically effective amount of NACA or diNACA sufficient to treat Niemann Pick Disease or Huntington's Disease. It will be understood that, as with the other embodiments defined above, NACA or diNACA is administered in daily doses of about 0.5 to 150 mg/Kg. In another aspect, NACA or diNACA is administered two or three times daily. In another aspect, NACA or diNACA is administered with a second active agent as disclosed above.

As used herein, “susceptible to” or “prone to” or “predisposed to” a specific disease or condition or the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200% or more.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” issued to indicate the presence of the recited integer (e.g., a feature, an element, a characteristic, a property, a method/process step or a limitation) or group of integers (e.g., feature(s), element(s), characteristic(s), property(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refer to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organization cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure.

Multiple inventions may be set forth according to the limitations of the multiple claims issuing from this disclosure, and such claims accordingly define the invention(s), and their equivalents, that are protected thereby. In all instances, the scope of such claims shall be considered on their own merits considering this disclosure but should not be constrained by the headings set forth herein.

All the compositions and/or methods disclosed and claimed herein can be made and executed without undue experimentation considering the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

### REFERENCES

Abdalkader M, Lampinen R, Kanninen K M, Malm T M, Liddell J R. Targeting Nrf2 to Suppress Ferroptosis and Mitochondrial Dysfunction in Neurodegeneration. Front Neurosci. 2018 Jul. 10; 12:466. doi: 10.3389/fnins.2018.00466. PMID: 30042655; PMCID: PMC6048292.

Andreasson et al. N-acetyl-L-cysteine improves survival and preserves motor performance in an animal model of familial amyotrophic lateral sclerosis. NeuroReport 2000 11: 2491-2493.

Ayton, S., et al., Parkinson's disease iron deposition caused by nitric oxide-induced loss of beta-amyloid precursor protein. J Neurosci, 2015. 35(8).

Che J, Lv H, Yang J, Zhao B, Zhou S, Yu T, Shang P. Iron overload induces apoptosis of osteoblast cells via eliciting ER stress-mediated mitochondrial dysfunction and p-eIF2α/ATF4/CHOP pathway in vitro. Cell Signal. 2021 August; 84:110024. doi: 10.1016/j.cellsig.2021.110024. Epub 2021 Apr. 24. PMID: 33901579.

Collaborators, G. M. N. D., Global, regional, and national burden of motor neuron diseases 1990-2016: a systematic analysis for the Global Burden of Disease Study 2016. Lancet Neurol, 2018. 17(12).

Devos, D., et al., A ferroptosis—based panel of prognostic biomarkers for Amyotrophic Lateral Sclerosis. Scientific Reports, 2019. 9(1).

Dixon S J, Lemberg K M, Lamprecht M R, Skouta R, Zaitsev E M, Gleason C E, Patel D N, Bauer A J, Cantley A M, Yang W S, Morrison B 3rd, Stockwell B R. Ferroptosis: an iron-dependent form of nonapoptotic cell death. Cell. 2012 May 25; 149(5):1060-72. doi: 10.1016/j.cell.2012.03.042. PMID: 22632970; PMCID: PMC3367386.

Do Van, B., et al., Ferroptosis, a newly characterized form of cell death in Parkinson's disease that is regulated by PKC. Neurobiol Dis, 2016. 94.

Fedida A, Ben Harouch S, Kalfon L, Abunassar Z, Omari H, Mandel H, Falik-Zaccai T C. Sedaghatian-type spondylometaphyseal dysplasia: Whole exome sequencing in neonatal dry blood spots enabled identification of a novel variant in GPX4. Eur J Med Genet. 2020 November; 63(11):104020. doi: 10.1016/j.ejmg.2020.104020. Epub 2020 Aug. 20. PMID: 32827718.

Fu R, Wassif C A, Yanjanin N M, Watkins-Chow D E, Baxter L L, Incao A, Liscum L, Sidhu R, Firnkes S, Graham M, Ory D S, Porter F D, Pavan W J. Efficacy of N-acetylcysteine in phenotypic suppression of mouse models of Niemann-Pick disease, type C1. Hum Mol Genet. 2013 Sep. 1; 22(17):3508-23. doi: 10.1093/hmg/ddt206. Epub 2013 May 10. PMID: 23666527; PMCID: PMC3736870.

Grolez, G., et al., The value of magnetic resonance imaging as a biomarker for amyotrophic lateral sclerosis: a systematic review. BMC Neurol, 2016. 16(1).

Guiney S J, Adlard P A, Bush A I, Finkelstein D I, Ayton S. Ferroptosis and cell death mechanisms in Parkinson's disease. Neurochem Int. 2017 March; 104:34-48. doi: 10.1016/j.neuint.2017.01.004. Epub 2017 Jan. 9. PMID: 28082232.

Han C, Liu Y, Dai R, Ismail N, Su W, Li B. Ferroptosis and Its Potential Role in Human Diseases. Front Pharmacol. 2020 Mar. 17; 11:239. doi: 10.3389/fphar.2020.00239. PMID: 32256352; PMCID: PMC7090218.

He R, Zheng W, Ginman T, Ottosson H, Norgren S, Zhao Y, Hassan M. Pharmacokinetic profile of N-acetylcysteine amide and its main metabolite in mice using new analytical method. Eur J Pharm Sci. 2020 Feb 15;143:105158. doi: 10.1016/j.ejps.2019.105158. Epub 2019 Nov. 16. PMID: 31740394.

Homma, T., et al., Edaravone, a free radical scavenger, protects against ferroptotic cell death in vitro. Exp Cell Res, 2019.

Ince, P. G., et al., Iron, selenium and glutathione peroxidase activity are elevated in sporadic motor neuron disease. Neuroscience Letters, 1994. 182(1).

Jenkins N L, James S A, Salim A, Sumardy F, Speed T P, Conrad M, Richardson D R, Bush A I, McColl G. Changes in ferrous iron and glutathione promote ferroptosis and frailty in aging Caenorhabditis elegans. Elife. 2020 Jul. 21; 9:e56580. doi: 10.7554/eLife.56580. PMID: 32690135; PMCID: PMC7373428.

Kumar P, Liu C, Hsu J W, Chacko S, Minard C, Jahoor F, Sekhar R V. Glycine and N-acetylcysteine (GlyNAC) supplementation in older adults improves glutathione deficiency, oxidative stress, mitochondrial dysfunction, inflammation, insulin resistance, endothelial dysfunction, genotoxicity, muscle strength, and cognition: Results of a pilot clinical trial. Clin Transl Med. 2021 March; 11(3):e372. doi: 10.1002/ctm2.372. PMID: 33783984; PMCID: PMC8002905.

Magtanong, L. and S. J. Dixon, Ferroptosis and Brain Injury. Dev Neurosci, 2018. 40(5-6).

Marin, B., et al., Clinical and demographic factors and outcome of amyotrophic lateral sclerosis in relation to population ancestral origin. Eur J Epidemiol, 2016. 31(3).

Masaldan S, Bush A I, Devos D, Rolland A S, Moreau C. Striking while the iron is hot: Iron metabolism and ferroptosis in neurodegeneration. Free Radic Biol Med. 2019 March; 133:221-233. doi: 10.1016/j.freeradbiomed.2018.09.033. Epub 2018 Sep. 25. PMID: 30266679.

Pansarasa, O., et al., SOD1 in Amyotrophic Lateral Sclerosis: “Ambivalent” Behavior Connected to the Disease. Int J Mol Sci, 2018. 19(5).

Pierzynowska K, Rintz E, Gaffke L, Wȩgrzyn G. Ferroptosis and Its Modulation by Autophagy in Light of the Pathogenesis of Lysosomal Storage Diseases. Cells. 2021 Feb. 10; 10(2):365. doi: 10.3390/cells10020365. PMID: 33578654; PMCID: PMC7916399.

Ren J X, Sun X, Yan X L, Guo Z N, Yang Y. Ferroptosis in Neurological Diseases. Front Cell Neurosci. 2020 Jul. 13; 14:218. doi: 10.3389/fncel.2020.00218. PMID: 32754017; PMCID: PMC7370841.

Simpson, E. P., et al., Increased lipid peroxidation in sera of ALS patients: a potential biomarker of disease burden. Neurology, 2004. 62(10).

Southon A, Szostak K, Acevedo K M, Dent K A, Volitakis I, Belaidi A A, Barnham K J, Crouch P J, Ayton S, Donnelly P S, Bush A I. CuII (atsm) inhibits ferroptosis: Implications for treatment of neurodegenerative disease. Br J Pharmacol. 2020 February; 177(3):656-667. doi: 10.1111/bph.14881. Epub 2020 Jan. 14. PMID: 31655003; PMCID: PMC7012947.

Spasic, S., et al., Edaravone May Prevent Ferroptosis in ALS. Curr Drug Targets, 2020. 21(8).

Statland J M, Barohn R J, McVey A L, Katz J S, Dimachkie M M. Patterns of Weakness, Classification of Motor Neuron Disease, and Clinical Diagnosis of Sporadic Amyotrophic Lateral Sclerosis. Neurol Clin. 2015 November; 33(4):735-48. doi: 10.1016/j.ncl.2015.07.006. Epub 2015 Sep. 8. PMID: 26515618; PMCID: PMC4629510.

Stockwell B R, Friedmann Angeli J P, Bayir H, Bush A I, Conrad M, Dixon S J, Fulda S, Gascón S, Hatzios S K, Kagan V E, Noel K, Jiang X, Linkermann A, Murphy M E, Overholtzer M, Oyagi A, Pagnussat G C, Park J, Ran Q, Rosenfeld C S, Salnikow K, Tang D, Torti F M, Torti S V, Toyokuni S, Woerpel K A, Zhang D D. Ferroptosis: A Regulated Cell Death Nexus Linking Metabolism, Redox Biology, and Disease. Cell. 2017 Oct. 5; 171(2):273-285. doi: 10.1016/j.cell.2017.09.021. PMID: 28985560; PMCID: PMC5685180.

Tang S, Gao P, Chen H, Zhou X, Ou Y, He Y. The Role of Iron, Its Metabolism and Ferroptosis in Traumatic Brain Injury. Front Cell Neurosci. 2020 Sep. 25; 14:590789. doi: 10.3389/fncel.2020.590789. PMID: 33100976; PMCID: PMC7545318.

Tanzi R E. The genetics of Alzheimer disease. Cold Spring Harb Perspect Med. 2012 Oct. 1; 2(10):a006296. doi: 10.1101/cshperspect.a006296. PMID: 23028126; PMCID: PMC3475404.

Tohgi, H., et al., Increase in oxidized NO products and reduction in oxidized glutathione in cerebrospinal fluid from patients with sporadic form of amyotrophic lateral sclerosis. Neurosci Lett, 1999. 260(3).

Wang, T., et al., Necroptosis is dispensable for motor neuron degeneration in a mouse model of ALS. Cell Death Differ, 2020. 27(5).

Wright et al N-Acetylcysteine improves mitochondrial function and ameliorates behavioral deficits in the R6/1 mouse model of Huntington's disease. Transl Psychiatry 2015 5, e492; doi:10.1038/tp.2014.131.

Yu, J., et al., Serum ferritin is a candidate biomarker of disease aggravation in amyotrophic lateral sclerosis. Biomed Rep, 2018. 9(4).

Zilka, O., et al., On the Mechanism of Cytoprotection by Ferrostatin-1 and Liproxstatin-1 and the Role of Lipid Peroxidation in Ferroptotic Cell Death. ACS Cent Sci, 2017. 3(3).

What is claimed is:

1. A method of treating or alleviating a disease, disorder, or condition associated with ferroptosis in a human subject that comprises:
administering to the human subject a therapeutically effective amount of N-acetylcysteine amide (NACA) or (2R,2R′)-3,3′-disulfanediyl bis(2-acetamidopropanamide) (diNACA) sufficient to reduce symptoms or treat the ferroptosis disease, disorder or condition.

2. The method of claim 1, wherein the NACA or diNACA is at least one of:
provided in or with a pharmaceutically acceptable carrier;
formulated for
administered dermally, orally, intravenously, intramuscularly, enterally, parenterally, topically, sublingually, rectally, or by inhalation, implant, or insert;
administered in daily doses of about 0.01, 0.1, 1, 5, 10, 20, 25, 30, 40, 50, 60, 70, 75, 80, 90, 100, 150, 150, 200, 250, 300, 333, 350, 400, 450, or 500 mg/Kg;
administered two or three times daily;
administered with a second active agent;
administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, propyl gallate, α-tocopherol, citric acid, ethylenediamine tetraacetic acid (EDTA), sorbitol, tartaric acid, or phosphoric acid; or
formulated for oral administration via a mini-tablet, capsule, tablet, effervescent, dual release, mixed release, sachet, powder, or liquid.

3. The method of claim 1, wherein a dose for administration is 0.01, 0.1, 1, 5, 10, 20, 25, 30, 40, 50, 60, 70, 75, 80, 90, 100, 150, 150, 200, 250, 300, 333, 350, 400, 450, 500, 600, 700, 750, 800, 900, 1,000, 2,500, 5,000, 7,500, or 10,000 mg per dose.

4. The method of claim 1, wherein the therapeutically effective amount decreases a loss of cognition or any physical ability by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, selected from at least one of 2 weeks, one month, 2 months, 3 months, 6 months, one year, 2 years, or 5 years.

5. The method of claim 1, wherein the disease, disorder, or condition associated with ferroptosis is declining cognition, cognitive impairment, declining physical function, physical decline, elevated inflammation, endothelial dysfunction, insulin resistance, central obesity, or loss of muscle mass (sarcopenia).

6. The method of claim 1, wherein the disease, disorder, or condition associated with ferroptosis is neurological diseases and disorders, neurodegeneration, stroke, including ischemic stroke and post-hemorrhagic stroke damage and neurotrauma, including traumatic brain injury (TBI).

7. The method of claim 1, wherein the disease, disorder, or condition associated with ferroptosis is Alzheimer's disease Huntington's disease, Niemann-Pick disease, Parkinson's disease, motor neuron disease or Sedaghatian-type spondylo-metaphyseal dysplasia.

8. The method of claim 1, wherein the disease, disorder, or condition associated with ferroptosis is Amyotrophic Lateral Sclerosis (ALS).

9. The method of claim 1, wherein the disease, disorder, or condition associated with ferroptosis is cancer, a breast cancer, a pancreatic cancer, colorectal cancer, lung cancer, liver cancer, glioma, ovarian cancer, neuroblastoma, head and neck cancer, melanoma, or esophageal cancer.

10. The method of claim 1, wherein the disease, disorder, or condition associated with ferroptosis is a kidney injury selected from ischemia-reperfusion and oxalic acid-induced kidney damage, rhabdomyolysis, or acute renal failure.

11. The method of claim 1, wherein the disease, disorder, or condition associated with ferroptosis is a mitochondrial dysfunction, osteoporosis, or a lysosomal storage disease.

12. The method of claim 1, wherein the disease, disorder, or condition associated with ferroptosis is cystinosis, Danon, Fabry, Krabbe, Gaucher, Niemann-Pick, Pompe, or Salla disease.

13. A method of treating or alleviating a disease, disorder, or condition associated with ferroptosis in a human subject that comprises:
identifying a human patient in need of treatment for ferroptosis or the disorder or condition associated with ferroptosis; and
administering to the human patient a therapeutically effective amount of N-acetylcysteine amide (NACA) or (2R,2R′)-3,3′-disulfanediyl bis(2-acetamidopropanamide) (diNACA) sufficient to reduce symptoms or treat the ferroptosis disease, disorder, or condition.

14. The method of claim 13, wherein the NACA or diNACA is at least one of:
provided in or with a pharmaceutically acceptable carrier;
administered dermally, orally, intravenously, intramuscularly, enterally, parenterally, topically, sublingually, rectally, or by inhalation, implant, or insert;
administered in daily doses of about 0.01, 0.1, 1, 5, 10, 20, 25, 30, 40, 50, 60, 70, 75, 80, 90, 100, 150, 150, 200, 250, 300, 333, 350, 400, 450, or 500 mg/Kg;
administered two or three times daily;
administered with a second active agent; or
administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, propyl gallate, α-tocopherol, citric acid, ethylenediamine tetraacetic acid (EDTA), sorbitol, tartaric acid, or phosphoric acid; or
formulated for oral administration via a mini-tablet, capsule, tablet, effervescent, dual release, mixed release, sachet, powder, or liquid.

15. The method of claim 13, wherein a dose for administration is 0.01, 0.1, 1, 5, 10, 20, 25, 30, 40, 50, 60, 70, 75, 80, 90, 100, 150, 150, 200, 250, 300, 333, 350, 400, 450, 500 600, 700, 750, 800, 900, 1,000, 2,500, 5,000, 7,500, or 10,000 mg per dose.

16. The method of claim 13, wherein the therapeutically effective amount decreases a loss of cognition or any physical ability by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, selected from at least one of 2 weeks, one month, 2 months, 3 months, 6 months, one year, 2 years, or 5 years.

17. The method of claim 13, wherein the disease, disorder, or condition associated with ferroptosis is declining cognition, cognitive impairment, declining physical function, physical decline, elevated inflammation, endothelial dysfunction, insulin resistance, central obesity, or loss of muscle mass (sarcopenia).

18. The method of claim 13, wherein the disease, disorder, or condition associated with ferroptosis is neurological diseases and disorders, neurodegeneration, stroke, including ischemic stroke and post-hemorrhagic stroke damage and neurotrauma, including traumatic brain injury (TBI).

19. The method of claim 13, wherein the disease, disorder, or condition associated with ferroptosis is Alzheimer's disease Huntington's disease, Niemann-Pick disease, Parkinson's disease, motor neuron disease or Sedaghatian-type spondylo-metaphyseal dysplasia.

20. The method of claim 13, wherein the disease, disorder, or condition associated with ferroptosis is Amyotrophic Lateral Sclerosis (ALS).

21. The method of claim 13, wherein the disease, disorder, or condition associated with ferroptosis is cancer, a breast cancer, a pancreatic cancer, colorectal cancer, lung cancer, liver cancer, glioma, ovarian cancer, neuroblastoma, head and neck cancer, melanoma, or esophageal cancer.

22. The method of claim 13, wherein the disease, disorder, or condition associated with ferroptosis is a kidney injury selected from ischemia-reperfusion and oxalic acid-induced kidney damage, rhabdomyolysis, or acute renal failure.

23. The method of claim 13, wherein the disorder or condition associated with ferroptosis is a mitochondrial dysfunction, osteoporosis, or a lysosomal storage disease.

24. The method of claim 19, wherein the disease, disorder, or condition associated with ferroptosis is cystinosis, Danon, Fabry, Krabbe, Gaucher, Niemann-Pick, Pompe or Salla disease.

25. A method of treating or alleviating a disease, disorder, or condition associated with ferroptosis comprising:
identifying a human in need of treatment for the disease, disorder, or condition associated with ferroptosis; and
administering to the human a therapeutically effective amount of N-acetylcysteine amide (NACA) or (2R,2R′)-3,3′-disulfanediyl bis(2-acetamidopropanamide) (diNACA) sufficient to treat the disorder or condition associated with ferroptosis, aging associated with declining cognition, cognitive impairment, declining physical function, physical decline, elevated inflammation, endothelial dysfunction, insulin resistance, central obesity, loss of muscle mass (sarcopenia), neurological diseases and disorders, including neurodegeneration, stroke, including ischemic stroke and post-hemorrhagic stroke damage, and neurotrauma, including traumatic brain injury, Alzheimer's disease, Huntington's disease, Niemann-Pick disease, Parkinson's disease, motor neuron disease, Amyotrophic Lateral Sclerosis, Sedaghatian-type spondylo-metaphyseal dysplasia, cancer, a breast cancer, a pancreatic cancer, colorectal cancer, lung cancer, liver cancer, glioma, ovarian cancer, neuroblastoma, head and neck cancer, melanoma, or esophageal cancer, kidney injuries including ischemia-reperfusion and oxalic acid-induced kidney damage, rhabdomyolosis and acute renal failure, mitochondrial dysfunction leading to osteoporosis, lysosomal storage diseases including cystinosis, Danon, Fabry, Krabbe, Gaucher, Niemann-Pick, Pompe, or Salla disease.
